(19) United States
(12) Patent Application Publication
(10) Pub. No.: US 2026/0061055 A1
(43) Pub. Date: Mar. 5, 2026

(54) METHODS OF PRODUCING EXPANDED NATURAL KILLER CELLS FROM CRYOPRESERVED APHERESIS SAMPLES AND USES THEREOF

(71) Applicant: KIADIS PHARMA INTELLECTUAL PROPERTY B.V., Amsterdam (NL)

(72) Inventors: Bruno FIGUEROA, Cambridge, MA (US); Alexandra GRELLA, Cambridge, MA (US); Sarah STURTEVANT, Cambridge, MA (US)

(21) Appl. No.: 19/314,479

(22) Filed: Aug. 29, 2025

Related U.S. Application Data
(60) Provisional application No. 63/689,439, filed on Aug. 30, 2024.

(51) Int. Cl. A61K 40/15; A61K 35/17; A61K 40/42; A61K 45/06; A61P 35/02; C07K 16/00; C12N 5/00; C12N 5/0783

(52) U.S. Cl. CPC A61K 40/15; A61K 35/17; A61K 40/42; A61K 45/06; A61P 35/02; C07K 16/00; C12N 5/0087; C12N 5/0646; C12N 2501/2321; C12N 2501/599

(57) ABSTRACT

Provided herein are methods of producing expanded natural killer cells from cryopreserved blood samples, compositions comprising natural killer cells produced by the methods, and uses thereof.

### CROSS REFERENCE TO RELATED APPLICATIONS

This application claims the benefit of, and relies on the filing date of, U.S. provisional patent application No. 63/689,439, filed 30 Aug. 2025, the entire contents of which are incorporated by reference herein.

### TECHNICAL FIELD

The disclosure relates to methods of producing expanded natural killer cells from frozen blood samples, particularly without the use of PBMC density gradient separation, pharmaceutical compositions comprising natural killer cells produced by the methods, and uses thereof.

### BACKGROUND

Blood is a critical component of medical care that can only be obtained from human donors. Following collection, whole blood samples are processed into various components such as plasma, platelets, and red cells. Red cell and platelet samples may be leuko-reduced to remove white cells from the samples to reduce the risk of a red cell recipient immunologically reacting to the sample after transfusion. Processed components of whole blood donations are then stored at room temperature in agitators (e.g., platelets), refrigerated temperatures about 6° C. (e.g. red cells), or are frozen (e.g., plasma and cryoprecipitated clotting factors). The shelf life of many of these blood components is limited such that platelets can only be safely stored for about five days, and refrigerated red cells are typically stored for only about 42 days. Frozen blood components, by comparison, can be stored for about one year. Frozen erythrocytes can be stored for about six days at refrigerated temperatures and then frozen in a cryoprotectant at minus 25° C. or colder temperatures for up to ten years before being thawed for use in transfusions.

Immune cells are in the forefront of cell-based therapies being developed to fight numerous medical conditions. Immune cells can be successfully isolated and modified from apheresis collections, such as those collected by leukopheresis. Leukopheresis is the process of extracting white blood cells (WBCs) from peripheral blood and then returning the remaining blood components back to the donor. A leukopak is an enriched apheresis product collected by leukopheresis, often with a targeted volume of about 150-400 mL total product volume and may contain five billion to as many as 20 billion WBCs. Leukopaks are used to obtain concentrated WBCs from which immune cells may be isolated and modified for use in cell-based therapies to treat diseases such as cancer and autoimmune diseases.

The convenience of freezing blood cells or blood components makes the use of frozen (cryopreserved) cells highly attractive for the manufacture of medicaments comprising immune cells for therapeutic purposes. For example, natural killer (NK) cells are cytotoxic members of the innate immune system that target cells that are malignant or compromised by infectious agents. Numerous groups have sought to employ NK cells as adoptive immunotherapeutic agents for clinical treatments, but they adhere to the accepted paradigm that immune cells must be isolated from blood sources that have never been frozen after apheresis collection. The prevailing view is that immune cells subjected to freeze-thaw processing will be compromised and therefore less effective as immunotherapeutic agents than immune cells obtained from fresh blood sources.

For example, one team reported that recovery of thawed cryopreserved NK cells was extremely poor after an overnight incubation in IL-2, and expanded NK cells failed to lyse K562 leukemia cells unless they were incubated overnight in IL-2. They concluded that it is better to use fresh sources of NK cells for in vivo human patient therapy over cryopreserved/thawed sources based on these observations with IL-2 activated NK cells. See Szmania et al., J. Immunother. (2015) 38(1):24-36. Hanley et al. (Molecular Therapy (2019) 27(7):1213-1214) compared fresh and frozen T-cell apheresis products and observed a decrease in cell viability within two days of thawing cryopreserved peripheral blood mononuclear cells (PBMCs), although they did not observe a difference in cell expansion efficiency. Damodharan et al. compared NK cell function from fresh apheresis samples with those of NK cells from donor collections cryopreserved after apheresis to assess whether cryopreservation diminishes NK cell activity. This group reported that NK cells from cryopreserved samples demonstrated decreased recovery of viable CD56⁺ cells, had reduced cytotoxicity, and generated less IFN-γ than did NK cells from fresh sources. Only fresh NK cells showed increased NKG2D levels, although both cell types produced increased granzyme B levels compared with NK cells pre-expansion. See Damodharan et al., Cytotherapy (2020) 22(8):450-457. Others suggest that cryopreservation of leukopheresis products followed by thawing with a declumping buffer before selection and expansion had a negligible effect on regulatory T cells (Treg cells) when compared to Treg cells selected and expanded from fresh samples. This team concluded that Treg cells generated from fresh apheresis samples are not significantly different (in terms of growth potential, immunosuppressive function, viability and phenotypic characterization) than those generated from cryopreserved apheresis products. See US 2019/0032013. This view was supported by the Facchin group, who reported no significant differences were recognized in clinical characteristics of patients, donors, and transplants between the cell transplants obtained from cryopreserved peripheral blood stem cells and fresh sources, although they acknowledged significantly reduced viability of CD34⁺ cells post thawing. See Facchin et al., J. Clin. Med. (2022) 11:4114.

Manufacturing off-the-shelf cell products for human therapy poses a number of challenges not faced at the research or “bench” level of cell isolation, expansion and dosage unit storage. For smaller batch production of cell products for certain immunotherapies and rare diseases, scale-out manufacture may suffice. Scale-out manufacture involves serial production employing multiple devices or workstreams run in parallel. Other cell therapeutics, such as pluripotent stem cells, require much larger commercial scale-up processes that increase the volume or number of cells generated through a single device or workstream to, for example, about 50 liters to perhaps even hundreds of liters. See Masri et al., Cell Gene Therapy Insights, (2017) 3)6): 447-467. Regardless of the scaling process followed to achieve high intensity cell production, exposure of human cells during production to mechanical and physiochemical stressors can alter characteristics of the desired final cell product, potentially impairing immune cell therapeutic effect. Such stressors may be induced by, inter alia, shear forces incurred during centrifugation, toxicity from metabolic waste generated in high density cell cultures, and intracellular ice crystal formation during freeze-thaw cycles. Many academic protocols use cell culturing systems to stimulate and expand immune cells to a desired clinical dose which enable high density cell growth without complex manipulations, permit nutrient exchange, and facilitate easy sampling. Unfortunately, these systems often require costly materials, stringent production environments, and highly trained staff to run, making them undesirable for commercially useful manufacturing. When scaling up cell manufacture to commercially viable levels, companies must balance such factors as final cell product consistency, quality, and quantity with manufacturing costs, workstream ease of operation, and compatibility with Good Manufacturing Practice requirements. Id. This is a daunting task to achieve in the highly regulated field of biological therapeutics.

As noted hereinabove, research has deemed immune cells, in particular NK cells, obtained from frozen human apheresis samples to be inferior—or at best equivalent to—apheresis samples that have never been frozen in the context of immunotherapeutic products. When combined with the challenges faced upon scaling-up human cell production, these factors put into doubt the ability to more efficiently produce at commercial scale therapeutic immune cell products from cryopreserved blood samples. Consequently, a need exists to better characterize the functionality of immune cells, particularly NK cells, obtained from frozen human apheresis samples to assess whether than are truly suitable for compositions to be formulated into products for the treatment of cancer and infectious diseases, and to efficiently and consistently manufacture these cell products at commercial scale.

### SUMMARY

Not all blood donations are alike, and therefore alloantigens and other factors in blood may complicate the process of making cell-based immunotherapeutic products. In alloimmunity, a person receiving transplanted allogeneic blood components may create antibodies against the transplanted materials. Alloimmune responses can result in the rejection of the transplanted material, which often results in loss of graft function. Allogeneic cell therapies (e.g., sourced from persons genetically distinct from the human recipient) are designed to rely on a single source of cells collected from a donor sample that can then be used to create cell populations that are processed into cellular drug products. Those cell-based drug products can be used to treat multiple patients. Accordingly, in lieu of testing multiple donors for compatibility with a specific patient, blood donors may be screened for suitable non-alloreactive genetic factors. Donors having those factors may then be identified as Universal Donors for the provision of blood for use in immunotherapeutic products.

Human natural killer (NK) cell function is controlled by surface inhibitory and activating receptors, including inhibitory and activating killer-cell immunoglobulin-like receptors (KIRs), whose genes vary in number and content between individuals. NK cells expressing inhibitory KIRs (iKIRs) for self-Human Leukocyte Antigen (HLA) class I molecules enables recognition of “self”, a process known as NK licensing. NK licensing enables NK cells to spare autologous cells from killing and elicit a cytotoxic response to targets lacking self-HLA Class I molecules. Consequently, utilization of NK cells in adoptive immunotherapy is highly restrained by the compatibility between an NK cell donor and the recipient of the donated NK cells. Thus, the “optimal donor,” referred to as a “Universal Donor,” is a donor who has an HLA and KIR expression profile allowing for maximal NK licensing and therefore the ability to treat a majority of patients.

An objective of Applicant's cell-based therapy research is to craft allogeneic off-the-shelf treatments that would enable rapid provision of bedside therapies in the clinical setting, thereby avoiding delays associated with matching a specific donor and a specific recipient patient, isolating blood components, and then processing blood cells only after a patient needing such treatment is identified by a hospitalist. By genotyping persons to identify Universal Donors whose blood components provide the maximum amount of anti-tumor alloreactivity toward a broad group of patients, sources of immune cells can be used that are best poised to attack tumor cells (e.g. graft versus leukemia effect) and other targets while reducing the risk of adverse events, such as initiating graft-versus-host-disease. Innovating techniques for the processing and storage of immune cells will leverage each sample provided by Universal Donors to benefit patients.

In furtherance of this objective, Applicant has pursued an NK cell therapy plan in which apheresis blood collection is obtained from Universal Donors and cryopreserved in blood centers. The cryopreserved blood samples are then processed ex vivo or in vitro to stimulate and exponentially expand CD56⁺ NK cells from peripheral blood mononuclear cells. These expanded NK cells are then formulated into cell-based therapy products (“drug products”), cryopreserved, and shipped to clinical treatment sites. When a patient is identified as needing such an NK cell therapy, the drug product will be readily available for immediate use. However, this approach requires frozen blood samples to be as effective as blood samples that have never been subjected to cryostorage. In an effort to improve the availability of Natural Killer (NK) cells for medical treatments, experiments were conducted to ascertain the suitability of frozen blood samples for these purposes and to improve the speed with which those frozen blood samples may be turned into cell-based immunotherapeutic products.

An embodiment of this NK cell therapy plan involves improvements to optimize the workflow directed to the manufacture of NK cell drug products. This workflow is exemplified by obtaining a Universal Donor apheresis sample that is either fresh or frozen. Initially, an apheresis sample is processed by removing PBMCs from the sample whereby red blood cells are isolated from WBCs by density gradient separation. The WBCs are then CD3⁺ depleted, stimulated by a membrane-bound IL-21, and then subjected to expansion of NK cells in cell culture. Cell culturing to facilitate expansion of the cell population may be repeated more than once. The cultured cells are then harvested, concentrated, and washed. Subsequently, the washed cells are formulated for cryopreservation in appropriate dosage units, and the resulting expanded NK cell drug product is frozen for shipment to a clinical care facility for use with patients.

While the above-noted embodiment is suitable for the manufacture of NK cell drug products, an effective, streamlined variation of the described workflow eliminates the use of density gradient separation of PBMCs for frozen apheresis samples, such as cryopreserved leukopak (a leukopheresis sample), which have substantially fewer red cells (about 1% to 5% RBCs) than does whole blood (40% to 50% RBCs). Density gradient separation is effective for isolating plasma and PBMCs from whole blood samples, but in commercial manufacturing the process can reduce the percent recovery of NK cells from blood samples, reduce the total NK cell number yield at the end of Day 0 of expansion culturing operations, and one Universal Donor unit is limited to seeding (initiating) one manufacturing campaign. Moreover, such density gradient separation modules often require greater than three hours to complete a separation module run in commercially scaled manufacturing settings. Elimination of density gradient separation reduces leukopak sample processing by two to three hours on Day 0 and subjects the blood cells to less manipulation (e.g., spinning through density gradient media) than when density gradient separation is employed. Other benefits include lower analytical in-process burden and higher NK cell recovery on Day 0 of the culturing process. By avoiding density gradient separation in the manufacturing workflow, NK cell expansion from frozen leukopaks becomes more efficient and achieves final NK cell population levels in a shorter time period. It is also possible to reduce the cost of goods sold (COGS) for final NK cell drug products by seeding (initiating) multiple manufacturing campaigns from a single Universal Donor collection.

In one embodiment, the disclosure provides a method of producing expanded natural killer (NK) cells from a frozen blood sample. The method comprises thawing a cryopreserved blood sample obtained from a subject or obtaining a thawed cryopreserved blood sample. The method further comprises obtaining peripheral blood mononuclear cells (PBMCs) from the thawed blood sample without the use of density gradient separation; depleting the PBMCs of CD3⁺ cells; culturing the PBMCs in a medium with membrane-bound IL-21 to expand NK cells contained in the PBMCs; and harvesting the expanded NK cells when population doubling reaches a threshold limit. That threshold limit may vary depending upon the initial concentration of cells to be expanded and the volume capacity of the vessel or bag in which cell culturing takes place. Specific threshold values may be donor dependent. Optimally, the total number of expanded natural killer cells obtained using this method may be at least ten billion (1×10¹⁰), at least one hundred billion (1×10¹¹), or at least about one trillion (1×10¹²).

In some aspects, disclosed is a method of the above-noted embodiment, wherein the total number of expanded NK cells obtained from a frozen source is greater than the total number of expanded control NK cells harvested from an equal starting number of cultured PBMCs obtained from a fresh blood sample obtained from the subject. Typically, a single blood sample is split into two aliquots for this purpose, with one aliquot being processed without freezing to serve as a control for the other aliquot which is cryopreserved. The method comprises obtaining PBMCs from the fresh blood sample using density gradient separation to remove red blood cells; depleting the PBMCs of CD3⁺ cells; culturing the PBMCs in a medium with membrane-bound IL-21 to expand NK cells contained in the PBMCs; and harvesting the expanded control NK cells when population doubling reaches a threshold limit.

In some aspects, the disclosed methods of the above-noted embodiments may further comprise a total number of expanded NK cells from a cryopreserved sample that is greater than the total number of expanded control NK cells after 10, 11, 12, or 13 days of culturing.

In some aspects, the present disclosure provides the method according to any of the above-noted embodiments, wherein harvesting is performed when a population doubling time reaches at least 40 hours.

In some aspects, the disclosure provides for the method of any of the above-noted embodiments, wherein the cryopreserved blood sample is a cryopreserved apheresis blood sample.

In some aspects, the disclosure provides a method according to any of the above-noted embodiments, wherein the membrane bound IL-21 is on a membrane particle. The membrane particle may further comprise 4-1BBL.

In some aspects, the present disclosure provides a method according to any one of the above embodiments, wherein the population doubling time of the expanded NK cells from a cryopreserved sample from Day 0 to Day 10 of culture is the same or less than the population doubling time of expanded control NK cells harvested from an equal starting number of cultured PBMCs obtained from a fresh blood sample obtained from the subject. The method comprises obtaining PBMCs from the fresh blood sample using density gradient separation to remove red blood cells; depleting the PBMCs of CD3⁺ cells; culturing the PBMCs in a medium with membrane-bound IL-21 to expand NK cells contained in the PBMCs; and harvesting the expanded NK control cells when population doubling reaches a threshold limit.

In some aspects, the present disclosure provides a method according to any of the above-noted embodiments, wherein the culturing step further comprises inclusion of IL-2 in the medium. The IL-2 may be added at a concentration of 100 IU/mL.

In some aspects, the present disclosure provides a method according to any of the above-noted embodiments, wherein upon harvesting the expanded NK cells have a viability of at least 70%. In other embodiments, expanded NK cell viability is at least 90%, and in some instances, it is at least 95%.

In some aspects, the present disclosure provides a method according to any of the above-noted embodiments, wherein the cytotoxicity of the harvested expanded NK cells from a cryopreserved sample is equivalent to the cytotoxicity of expanded control NK cells harvested from an equal starting number of cultured PBMCs obtained from a fresh blood sample obtained from the subject. The control NK cells are subjected to a method comprising obtaining PBMCs from the fresh blood sample using density gradient separation to remove red blood cells; depleting the PBMCs of CD3⁺ cells; culturing the PBMCs in a medium with membrane-bound IL-21 to expand NK cells contained in the PBMCs; and harvesting the expanded NK cells when population doubling reaches a threshold limit. The cytotoxicity may be measured as percent killing of target cancer cells exposed to the harvested expanded NK cells.

In some aspects, the present disclosure provides for a method according to the above-noted embodiments, wherein the NK cells are exposed to plasma membrane-bound IL-21 (PM21) particles multiple times during the culturing step.

In some aspects, the present disclosure provides for a population of expanded natural killer cells produced by the methods of the above-noted embodiments.

In some embodiments, the population comprises expanded natural killer cells obtained from a cryopreserved blood sample that exhibit equivalent percentages of CD16⁺, CD314⁺, and CD355⁺ cell surface markers as expanded natural killer cells obtained from a fresh blood sample.

In some such aspects, the present disclosure provides for a pharmaceutical composition comprising an effective amount of the expanded natural killer cells produced by the method of any of the above-noted embodiments and a pharmaceutically acceptable carrier.

In some aspects, the pharmaceutical composition of the above-noted embodiments further comprises an anti-infective agent or an anti-cancer agent. The anti-cancer agent may be selected from an antibody, a chemotherapeutic drug, and an immunotherapeutic agent. The anti-infective agent may be selected from an antiviral agent, an antibacterial agent, and an antifungal agent.

In some aspects, the present disclosure provides for a method of treating an infection or cancer in a subject in need thereof comprising administering to the subject an effective amount of the pharmaceutical composition of any one of the above-noted embodiments.

In some aspects, the present disclosure provides for a pharmaceutical composition of any one of the above-noted embodiments for use in the treatment of an infection or cancer in a subject.

In some aspects, the present disclosure provides for the use of expanded natural killer cells in the manufacture of a medicament for treating an infection or cancer comprising thawing a cryopreserved blood sample obtained from a subject; obtaining peripheral blood mononuclear cells (PBMCs) from the thawed blood sample without the use of density gradient separation; depleting the PBMCs of CD3⁺ cells; culturing the PBMCs in a medium with membrane-bound IL-21 to expand NK cells contained in the PBMCs; and harvesting the expanded NK cells when population doubling reaches a threshold limit. The culturing step may further comprise inclusion of IL-2 in the medium. The IL-2 may be added to the culture medium at a concentration of 100 IU/mL.

In some aspects, the disclosure provides for the use of any of the above-noted embodiments further comprising harvesting being triggered when population doubling time reaches 40 hours.

In some aspects, the disclosure provides for the use of any of the above-noted embodiments, wherein the membrane-bound IL-21 is provided by plasma membrane-bound IL-21 (PM21) particles, exosomes comprising membrane-bound IL-21 (EX21 exosomes), or feeder cells comprising membrane-bound IL-21 (FC21 feeder cells). The vesicles providing the membrane-bound IL-21 may be either EX21 exosomes comprising 41-BBL or PM21 particles comprising 41-BBL, or both. Preparation of PM21 particles, EX21 exosomes, and FC21 feeder cells can be performed using techniques known in the art.

In some embodiments, the disclosure provides for the use of any of the above-noted embodiments, wherein the total number of expanded NK cells harvested from cultured thawed cryopreserved PBMCs exceeds the number of expanded NK cells harvested from an equal starting number of cultured fresh PBMCs subjected to the depleting, culturing and harvesting steps.

In some aspects, the disclosure provides for a use of any of the above-noted embodiments, wherein upon harvesting the expanded NK cells have a viability of at least 70%, at least 90%, or at least 95%.

In some aspects, the method or use of any of the above-noted embodiments may utilize a cryopreserved blood sample obtained from a leukopheresis pack.

In some aspects, the disclosure provides a use of any of the above-noted embodiments, wherein the expanded NK cells are combined with a pharmaceutically acceptable carrier to generate a medicament comprising NK cells formulated for treating an infection or cancer.

In some aspects, the disclosure provides for a kit comprising a pharmaceutical composition as described in any of the above-noted embodiments and instructions for use according to the methods of any of the above-noted embodiments.

### BRIEF DESCRIPTION OF THE DRAWINGS

FIG. 1A-D shows the number of natural killer cells expanded from portions of an apheresis donation that was split into a fresh (i.e., never frozen) leukopak sample and a frozen leukopak sample. Bags of frozen cells were thawed into pre-warmed PBS/EDTA/HAS in a BSC and directly loaded onto an automated cell processing instrument. T-cell levels remained below 0.1% for the entire expansion run and cell viability remained constant. Natural killer cells were harvested from expansion cultures when the population doubling time rose above 40 hours Monitoring the population doubling time is beneficial for, amongst other reasons, ensuring that the maximum capacity of the 10 L culture bags was not exceeded.

FIG. 2A-C shows the criteria associated with cell product release assays that characterize the phenotypes and functionalities of expanded natural killer cells. NK cells expanded from cryopreserved (frozen) samples were harvested on Day 12 of culture when the population doubling time rose above 40 hours. 34.4×10⁹ NK cells were harvested from the 10 L bags in which cryopreserved samples were expanded. NK cells from fresh (never frozen) apheresis samples were harvested on Day 13 of culture when their population doubling time rose above 40 hours. A total of 20.7×10⁹ NK cells were harvested from 10 L culture bags in which the fresh samples were cultured. Release assays were run on fresh NK cells at the pre-harvest, formulation, and cryopreservation process stages as well as on thawed and rested drug product NK cells. Harvested NK cells were processed in an automated cell concentration and washing instrument and then frozen down in 5 ml vials and CS50 Drug Product bags at dose in formulation media.

FIG. 3A-B provides metrics measured upon harvesting expanded NK cells from fresh and frozen apheresis samples.

FIG. 4A-B shows the results of a comparison of expanded NK cells cultured from fresh and frozen apheresis samples obtained from multiple donors. Blood donations from four human donors were split and processed into fresh and cryopreserved samples. Samples from two donors were expanded from Day 0 to Day 7 of culture. Samples from the remaining two donors were subjected to expansion culturing from Day 0 through Day 13.

FIG. 5A-D shows another compilation of harvest data, cytotoxicity data, characterization data, and release data for a comparison study of NK cell expansion from fresh (never frozen) apheresis samples and cryopreserved (frozen) apheresis samples with n=11 split donations tested.

FIG. 6A-B shows graphically how the elimination of density gradient separation (No DGS) of peripheral blood mononuclear cells from apheresis samples enables the seeding of multiple cell-based drug product manufacturing campaigns.

FIG. 7A-E shows that NK cells expanded from apheresis samples (frozen leukopaks) not subjected to density gradient separation (No DGS) are not functionally different from NK cells expanded from apheresis samples (frozen leukopaks) that were subjected to DGS. Both categories of NK cells exhibit similar growth kinetics.

FIG. 8A-B shows in tabular form that the elimination of density gradient separation from NK cell manufacturing processes using frozen leukopaks does not impact NK cell drug products in terms of NK cell release specifications and harvest metrics.

FIG. 9A-B graphically compares NK cells expanded from apheresis samples (frozen leukopaks) subjected to density gradient separation and not subjected to density gradient separation measuring indicia of NK cell phenotypic and functionality characteristics.

FIG. 10 shows in chart form that various white blood cell markers were similar in apheresis samples subjected to density gradient separation during processing (DGS Reference) compared to apheresis samples that were not subjected to density gradient separation during processing (PD runs 1-3).

### DETAILED DESCRIPTION

### Definitions

Unless otherwise noted, the terms used herein have definitions as ordinarily used in the art. Some terms are defined below, and additional definitions can be found within the rest of the detailed description.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “in some embodiments,” “in certain embodiments,” “in other embodiments,” “in some other embodiments,” or the like, refers to embodiments of all aspects of the disclosure, unless the context clearly indicates otherwise.

As used herein, the terms “treat,” “treating” or “treatment of” (and grammatical variations thereof) mean that the severity of the subject's condition is reduced, at least partially improved or stabilized and/or that some alleviation, mitigation, decrease or stabilization in at least one clinical symptom is achieved and/or there is a delay in the progression of the disease or disorder.

As used herein, the terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the compositions and/or methods described herein. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the compositions and/or methods described herein.

As used herein, a “therapeutically effective amount” is the amount of a pharmaceutical composition provided herein that is effective to treat or prevent a disease or disorder in a subject or to ameliorate a sign or symptom thereof. The “therapeutically effective amount” may vary depending, for example, on the disease and/or symptoms of the disease, severity of the disease and/or symptoms of the disease or disorder, the age, weight, and/or health of the patient to be treated, and the judgment of the prescribing physician.

As used herein, an “effective” amount generally means an amount which results in a desired effect in a given system. The effect may be achieved in an in vitro or ex vivo system, such as the stimulation of cytokine formation. Or the effect may be in vivo, such as an amount sufficient to effectuate a localized or systemic response.

As used herein, the term “formulated” refers to the multistep process in which active substances, such as an active pharmaceutical ingredient, are combined with other components to produce a final medicinal product. The process takes into consideration various factors, including particle size, polymorphism, pH, solubility, and the like. Pharmaceutical formulations can result in compositions taking various dosage forms designed for particular routes of administration, such as oral, intravenous, intramuscular, transdermal, topical, and inhalational.

As used herein, the term “drug product” refers to a finished dosage form that contains one or more active ingredients generally—but not necessarily—in combination with one or more inactive ingredients. The active ingredient may be a chemical or biological substance that is intended to furnish pharmacological activity or other direct effect in the diagnosis, cure, mitigation, treatment, or prevention of disease. Drug products may be manufactured in many dosage forms, including tablets, capsules, aerosols, liquids, gels, lozenges, and patches.

As used herein, the term “allogeneic” means material derived from individuals of the same species that are sufficiently unlike genetically to interact antigenically. Allogeneic therapies can increase the risk of eliciting an immune response within a recipient patient.

Ranges may be expressed herein as from “about” one particular value, and/or to “about” another particular value. When such a range is expressed, also specifically contemplated and considered disclosed is the range-from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

The section headings used herein are for organizational purposes only and are not to be construed as limiting the subject matter described.

Referring now to the Figures wherein like numbered features shown therein refer to like elements throughout unless otherwise noted. The present disclosure relates generally to a method of producing expanded natural killer cells from a cryopreserved blood sample obtained from a subject donor, and more specifically, to provide an efficient method of processing natural killer cells from a cryopreserved blood sample without the use of density gradient separation.

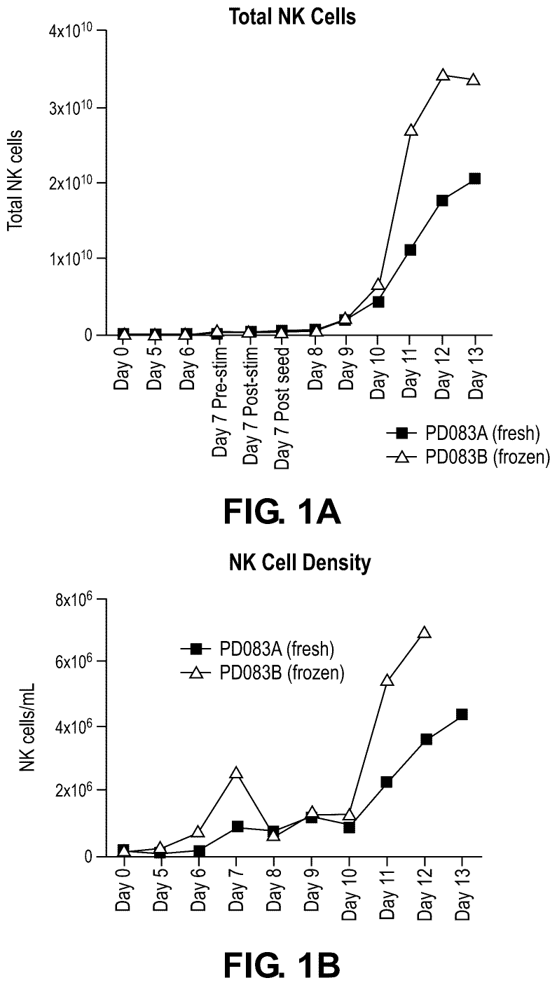

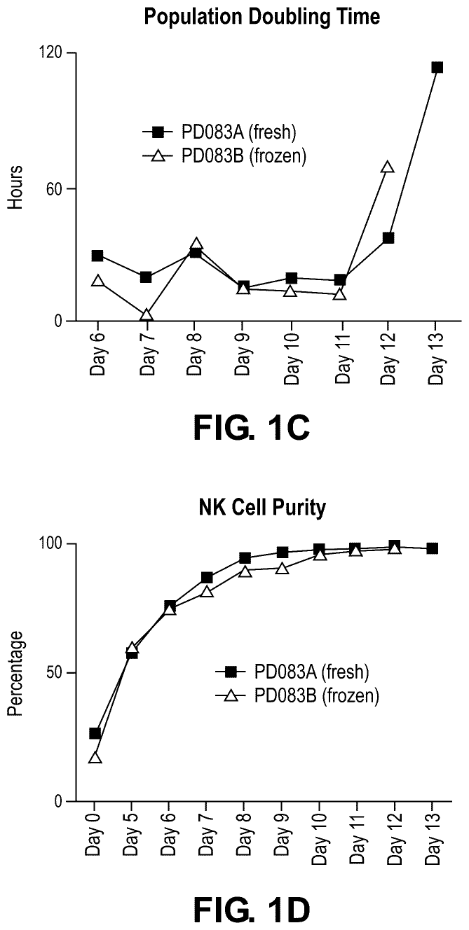

FIG. 1A graphically depicts the total number of NK cells achieved during expansion of fresh leukopak samples and frozen leukopak samples spanning from Day 0 through Day 13 of culture. FIG. 1B depicts the cell density of NK cells from fresh and frozen leukopak samples through Day 13 of the expansion culturing process. FIG. 1C shows the population doubling time for NK cells expanded from fresh and frozen leukopak samples. FIG. 1D depicts the NK cell purity levels achieved between Day 0 and Day 13 of the expansion of fresh and frozen leukopak samples. Surprisingly, the data indicate that frozen apheresis samples produced more total natural killer cells upon expansion than do apheresis samples that have never been frozen. Expanded NK cells from frozen leukopak samples reached higher density levels and their population doubling time peaked earlier than was observed for NK cells expanded from fresh samples. No differences in the purity of the expanded NK cell populations were observed from both fresh and frozen samples during the expansion period.

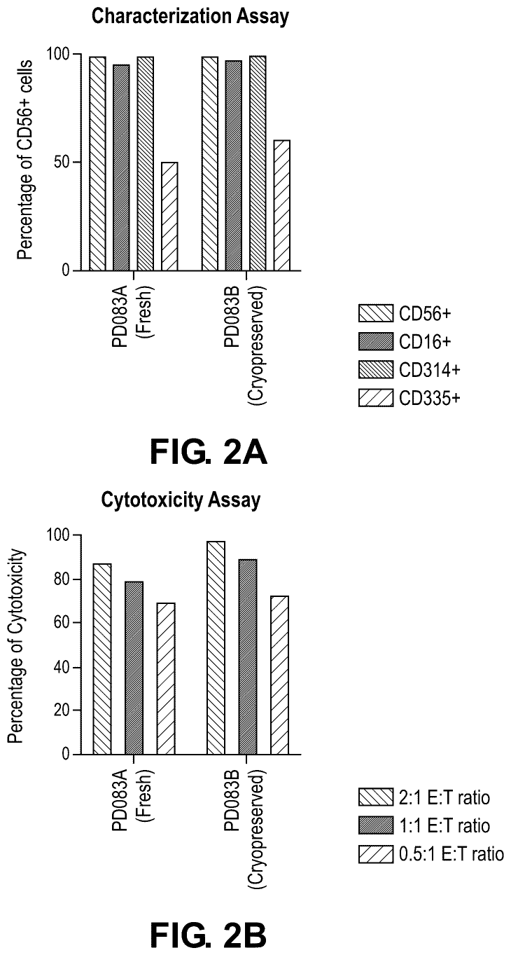

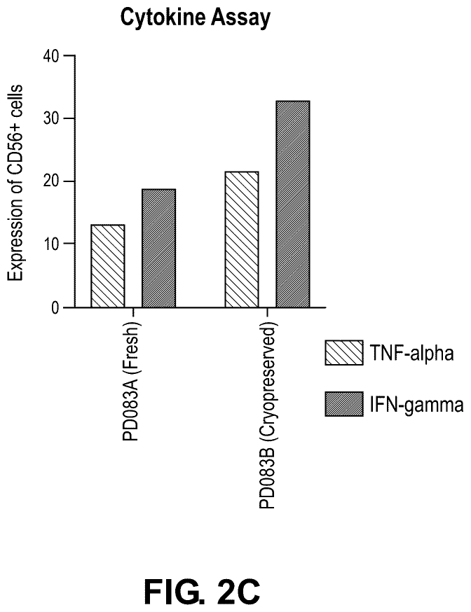

FIG. 2A shows the characteristics of expanded NK cells obtained from fresh and cryopreserved apheresis samples. FIG. 2A depicts the relative expression of markers CD56⁺, CD16⁺, CD314⁺, and CD335⁺ on expanded NK cells harvested from fresh and cryopreserved culture samples. The expression of these markers did not differ between fresh and cryopreserved samples. The cytotoxicity of the expanded NK cell populations cultured from fresh and cryopreserved samples were substantially the same, as shown in FIG. 2B. FIG. 2C demonstrates that cytokines Tumor Necrosis Factor-Alpha (TNF-α) and Interferon-gamma (IFN-γ) were produced at a slightly higher levels in NK cells expanded from cryopreserved samples than in NK cells expanded from fresh samples.

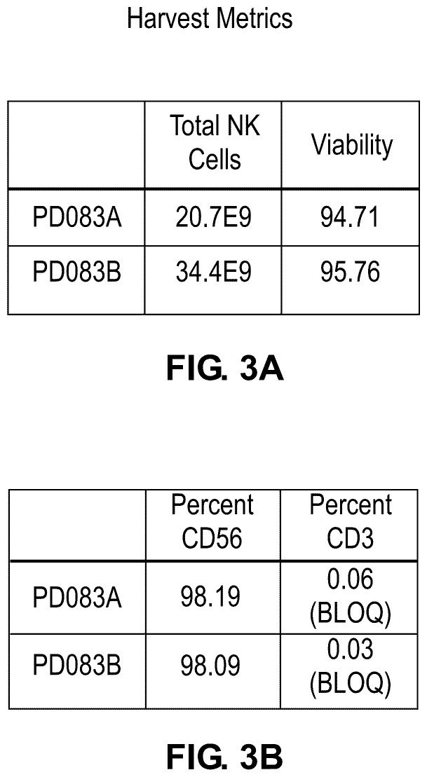

FIGS. 3A and 3B demonstrate that while the total number of expanded NK cells harvested from cultured frozen samples (34.4×10⁹ NK cells) exceeded the total from cultured fresh samples (20.7×10⁹ NK cells), cell viability was essentially the same between the two NK cell populations. Likewise, the percentages of CD56⁺ cells and CD3⁺ cells were essentially the same between the two groups as well.

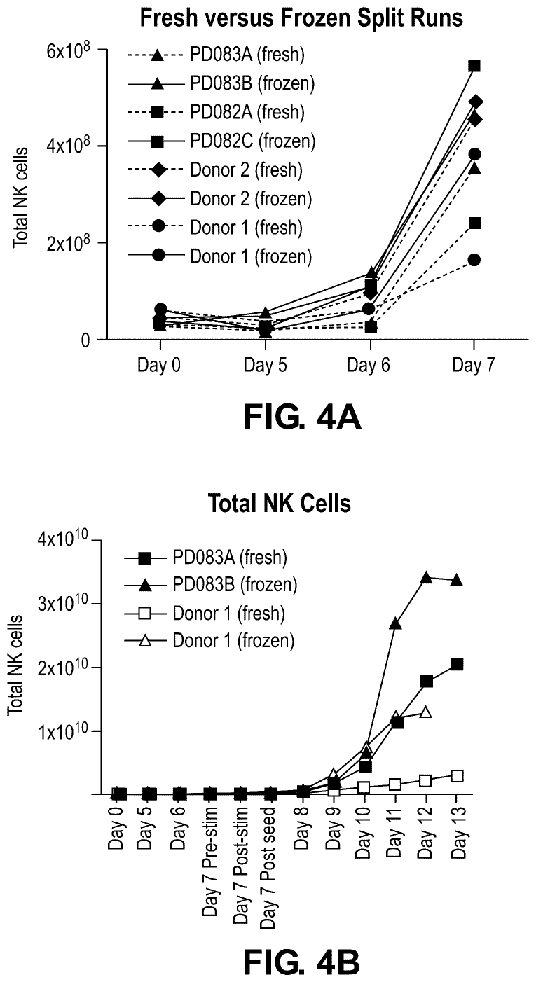

FIG. 4A shows that the total number of expanded NK cells obtained though Day 7 of culturing was higher from cryopreserved samples than from fresh samples. FIG. 4B shows that this superiority of NK cells expanded from frozen samples continued through Day 13 of expansion culturing for two donors. Thus, cryopreserved apheresis samples consistently yielded more NK cells upon expansion than did fresh apheresis samples.

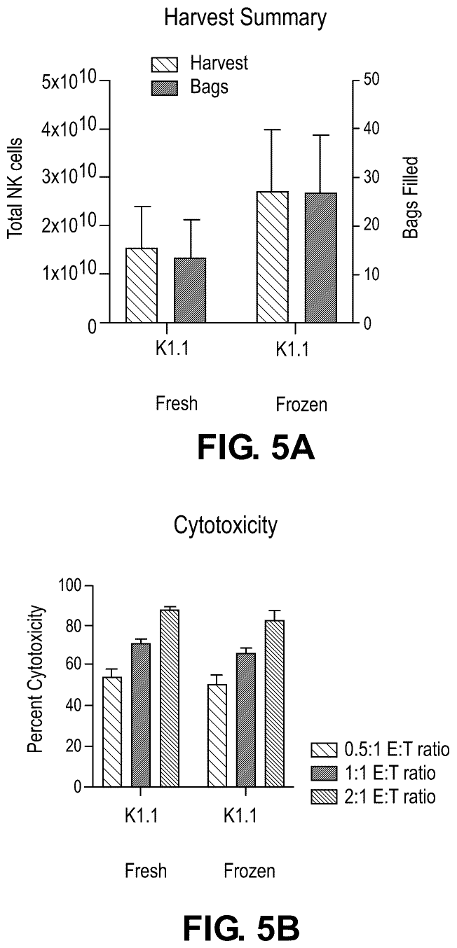

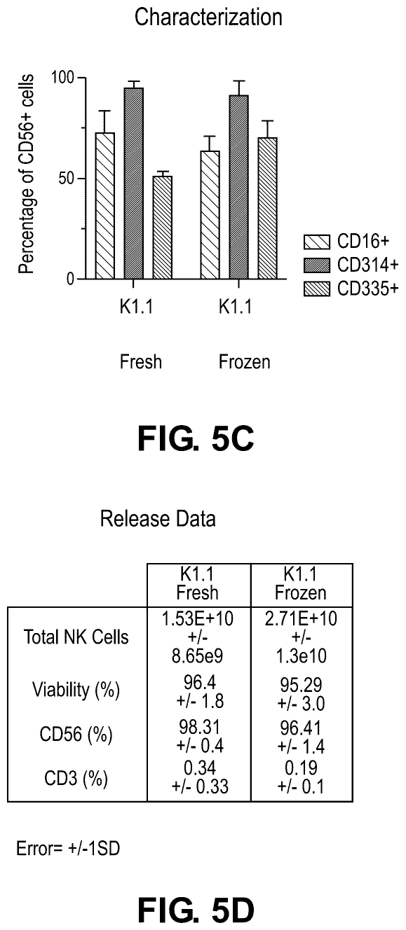

FIG. 5A shows that cryopreserved apheresis samples yield higher levels of expanded NK cells and Drug Product bags than do fresh apheresis samples. FIG. 5B demonstrates that NK cells expanded from cryopreserved apheresis samples exhibit cytotoxicity equivalent to that of NK cells expanded from fresh apheresis samples. FIG. 5C shows that the phenotype of NK cells expanded from cryopreserved apheresis samples is equivalent to that of NK cells expanded from fresh apheresis samples. FIG. 5D depicts in tabular format that total NK cell yield, percent NK cell viability, percent CD56 expression and percent CD3 expression are equivalent between fresh and frozen apheresis samples.

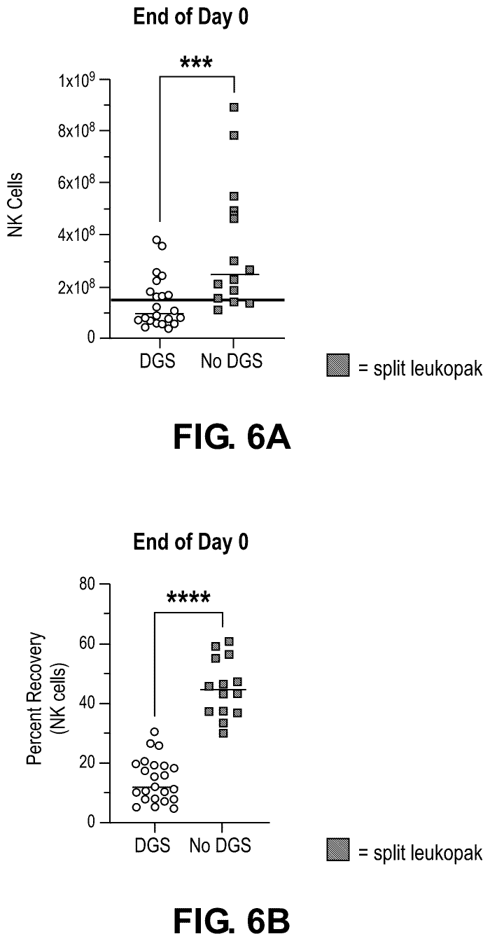

FIG. 6A-6B shows that at the end of Day 0 of the manufacturing process, the target seed levels of NK cells from samples not subjected to DGS (blue squares, split leukopaks, n=14) were achieved more consistently than for apheresis samples subjected to DGS (red circles, n=23). In addition, the percentage recovery of NK cells at the end of Day 0 was much higher for apheresis samples not subjected to DGS than for apheresis samples subjected to DGS.

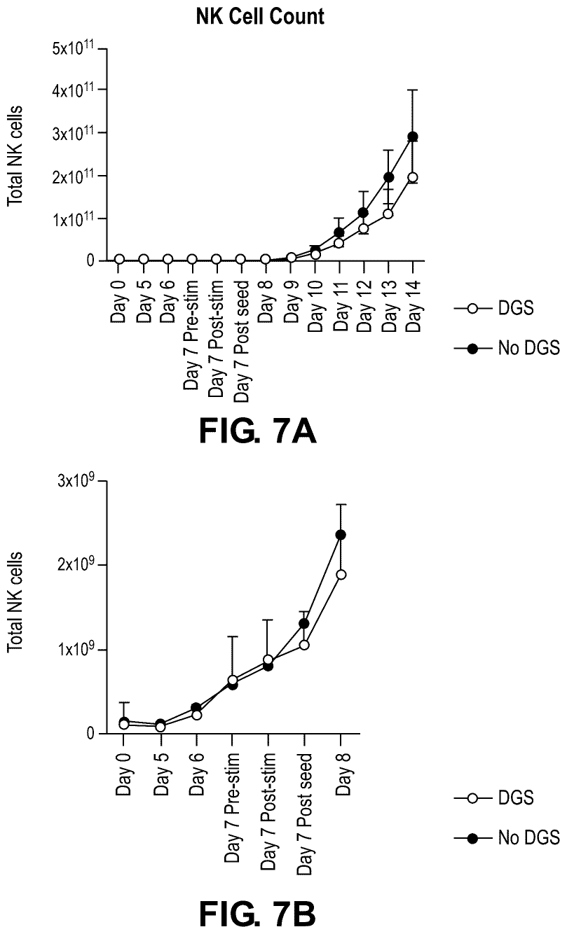

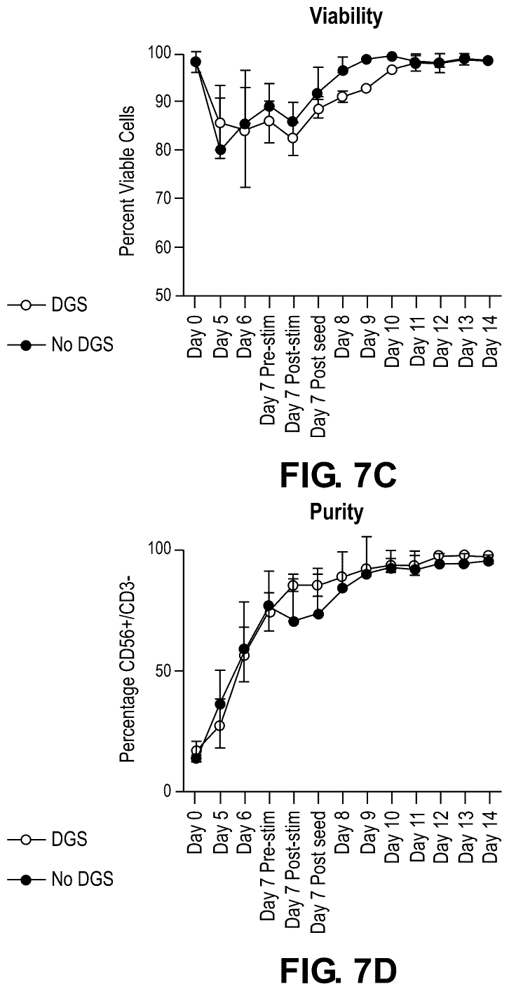

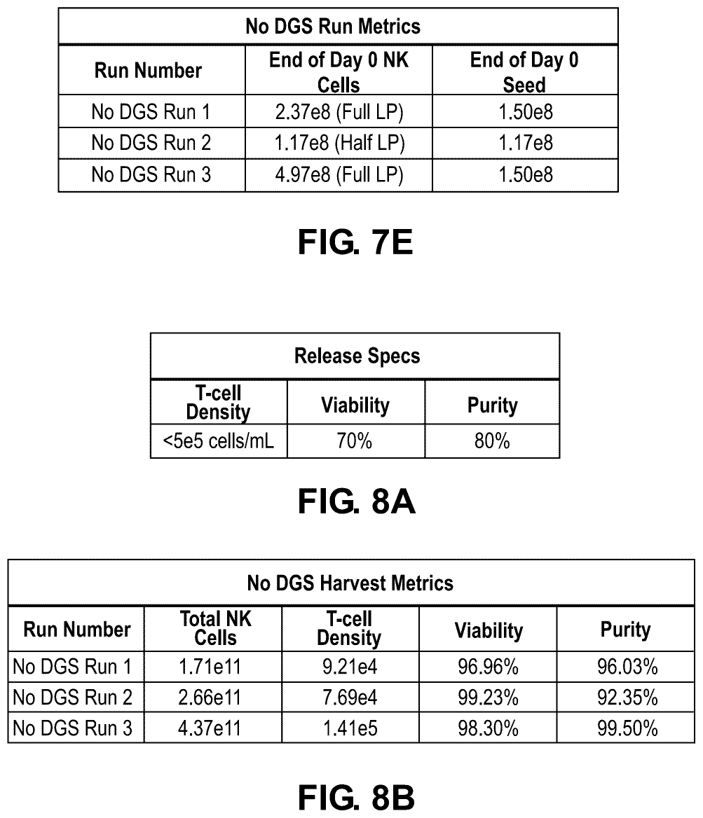

FIGS. 7A-7E demonstrate that total NK cell count through Day 14 of expansion culturing, the percent viability of NK cells obtained, and the purity of the NK cells obtained are remarkably alike between NK cells expanded from apheresis samples not subjected to density gradient separation (DGS) as depicted in blue and NK cells expanded from apheresis samples that were subjected to DGS as depicted in red. The functionality of the two sets of NK cells are not meaningfully different.

FIGS. 8A-8B present in tabular format the release specification and the harvest metrics measure for NK cell drug product prepared according to the streamlined manufacturing workflow protocol. The label “No DGS” refers to cells processed without a density gradient separation step. The purity and NK cell viability values were consistent across the samples assessed.

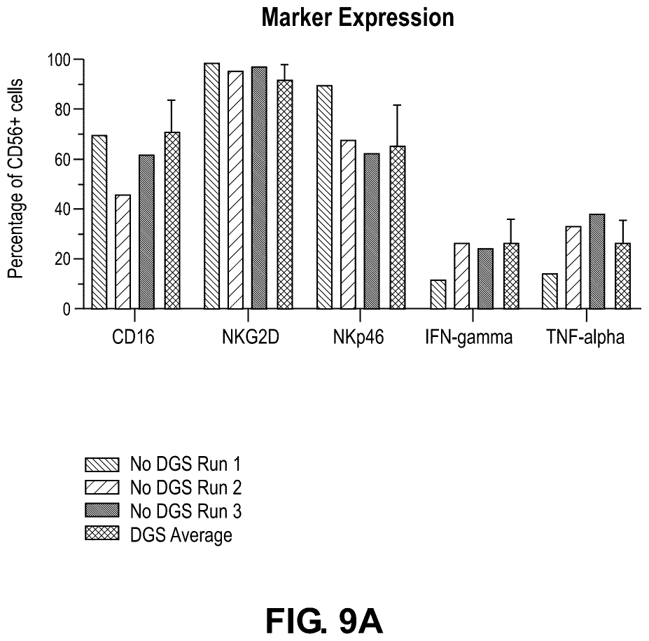

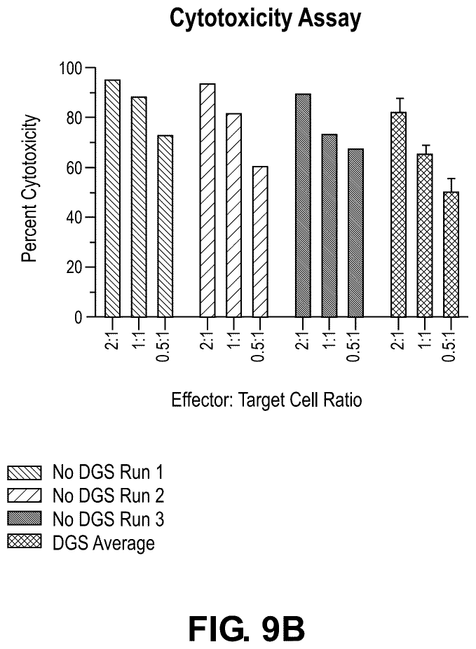

FIG. 9A compares the expression of markers CD16, NKG2D, NKp46, interferon-gamma, and tumor necrosis factor-alpha relative to the percentage of CD56⁺ cells in the test samples. The data indicate that elimination of density gradient separation from apheresis sample processing does not affect NK cell phenotype. FIG. 9B depicts the results of NK cell cytotoxicity assays run on NK cells expanded from apheresis samples subjected to density gradient separation during processing and NK cells that did not. The data show that both groups of NK cells exhibited the same functionality as measured by cytotoxicity.

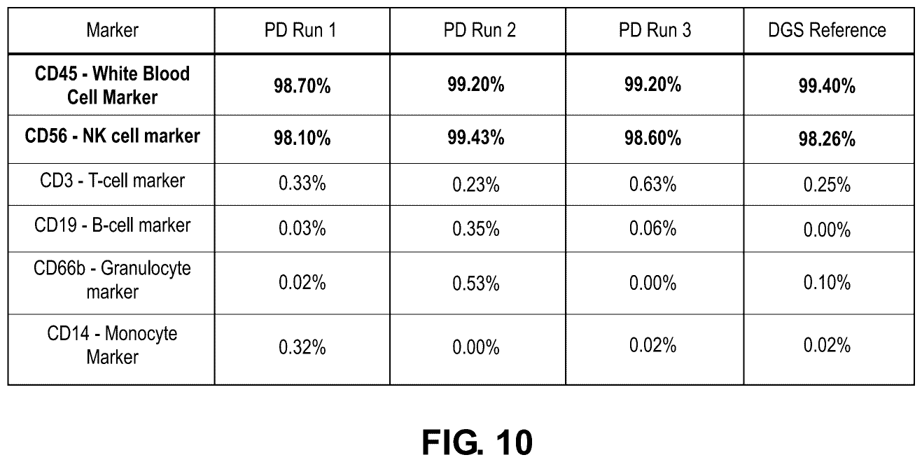

FIG. 10 presents white blood cell markers detected in NK cells manufactured using density gradient separation during process development (PD runs 1-3) in comparison to reference values obtained from NK cells processed from frozen apheresis samples without using density gradient separation. Most strikingly, the levels of CD45 (a white blood cell marker) and CD56 (an NK cell marker) were essentially uniform across all of the test groups and the DGS Reference.

Improving manufacturing processes for cell-based immunotherapies is a continuing goal for pharmaceutical and biotechnology companies. Herein are described streamlined manufacturing process conditions under which cryopreserved apheresis samples surprisingly led to more total NK cells at harvest than were harvested from fresh apheresis samples from the same donor, as shown in FIG. 4. The data demonstrate that, contrary to conventional thinking, NK cells can be successfully expanded from frozen human apheresis samples concluding with on average 4.04×10¹⁰ NK cells (±5.3×10⁹) at harvest with a reduced processing time. Eliminating density gradient separation during processing surprisingly enabled a greater NK cell yield for initial seeding, which led to the ability to seed at a larger scale. This in turn resulted in increased NK cell “drug substance” per run, and multiple runs could be achieved per Universal Donor collection. Unexpectedly, the fresh and cryopreserved apheresis NK cell drug product lots performed comparably in release assays. Analytics demonstrated equivalent potency using cytotoxicity as a surrogate metric and NK cell characterization for cells obtained from frozen apheresis samples compared with NK cell from fresh apheresis samples as a starting material. And frozen apheresis samples surprisingly reduced donor-related NK cell drug product batch variability that is associated with NK cell drug products derived from fresh apheresis samples (success rate: >90% for frozen apheresis derived products versus 70% for fresh apheresis derived products). Utilization of frozen apheresis samples confers benefits on supply chain logistics and reduces Cost of Goods Sold (COGs). Such benefits include manufacturing flexibility (timing and geographic), as well as flexibility through product inventory creation. NK cell drug product manufacturing campaign starts are de-risked due to donor deferrals through inventory creation. This opens the potential for multiple manufacturing campaigns from a single Universal Donor, thereby reducing the burden on the Universal Donor network and improving manufacturing efficiency on a commercial scale.

The disclosure will be further clarified by the following examples, which are intended to be purely exemplary of the disclosure and in no way limiting.

### EXAMPLES

### Example 1: Processing Natural Killer Cells From Blood Samples

### PBMC Sourcing

Peripheral leukopheresis donations from healthy individuals were obtained from Charles River Laboratories or Oklahoma Blood Institute. All donors were screened for CMV positivity and infectious disease panel. Collections were shipped at 4° C. overnight for next day processing or were cryopreserved at the collection site and then shipped and stored in liquid nitrogen (LN2) for later use.

### PBMC Processing, In-Process Analysis, and CD3 Depletion

The CliniMACS Prodigy® Automated Cell Processing System is utilized for GMP-compliant expansion of Natural Killer (NK) cells. On day 0, cryopreserved leukopheresis packs underwent a controlled thaw using a Barkey Plasmatherm™ Cell and Gene dry thawing system. For every 3E9 nucleated cells available, thawed material was diluted with 70 mL of 37° C. CliniMACS® PBS/EDTA Buffer supplemented with HSA to 1%. Diluted starting material was passed through a Pall SQ40™ Blood Transfusion Filter and loaded into the Prodigy Centricult™ Unit (CCU) of a TS520 tubing set. PBMCs were then further washed with PBS/EDTA buffer supplemented with HSA to 0.5% and centrifuged to remove platelet content. When fresh leukopheresis material was being processed, density gradient separation (DGS) was performed with Ficoll-Paque™ density gradient media to remove red blood cells. With cryopreserved leukopheresis, the DGS processing step was omitted from the protocol.

When utilizing fresh leukopheresis, a sample was acquired from the CCU post-density gradient separation. With cryopreserved leukopheresis, when the DGS was skipped, a sample was acquired after passing the material through the Pall Blood Transfusion Filter. Total cell counts are determined by flow cytometry.

Cells in the CCU were blocked by addition of Intravenous Immunoglobulin (IVIG) to 0.125%. Cells were then incubated with anti-CD3 iron-conjugated microbeads for 30 minutes. Post-incubation, cellular material was sequentially passed over the magnetic column in the TS520 tubing set, 600×10⁶ CD3+ cells at a time. The total number of column passes was determined by the in-process analysis described below. During CD3 depletion, PBMCs were transferred to an external cell bag on the TS520 tubing set. The column and CCU were washed with sterile water and then PBS/EDTA/HSA buffer to eliminate any remaining T-Cells. The depleted PBMCs were passed over the column again to capture residual CD3+ cells and to transfer the cells back into the CCU.

### Culturing NK Cells (Day 0 to 7)

CD3-depleted MNCs were exchanged out of PBS/EDTA buffer into completed GMP SCGM Media in the Prodigy Centricult™ Unit. Completed SCGM (cSCGM) was made by supplementing heat-inactivated, gamma-irradiated FBS to 10%, Gibco GlutaMAX™ supplement to 1% and adding IL-2 to 100 IU/mL. The cells were then eluted from the Prodigy and a sample taken for flow cytometry analysis as described below. Post processing, a maximum of 1.50×10⁸ NK cells were loaded into an automated cell processing system with a TS620 tubing set installed on it and subjected to media exchanges to reduce residual PBS/EDTA buffer in the culture. NK cells were stimulated by addition of 25 mg membrane bound IL-21 (PM21) particles per 3×10⁷ NK Cells loaded into the Prodigy Centricult™ Unit (CCU). PM21 particles were incubated with cells for 15 minutes in a volume of 100 mL before fresh cSCGM supplemented with IL-2 (100 IU/mL) was added to the final culture volume to reach a cell concentration of 3×10⁵ NK cells/mL. The cell culture was maintained without agitation at 37° C. and 5% CO2.

On days 4, 5, and 6, an 80% media exchange was performed, supplying fresh cSCGM. Culture conditions were maintained at 37° C. and 5% CO2 with agitation: 75 rpm spinning for 5 seconds every 5 minutes for days 4 through 7. On days 5, 6, and 7, samples were taken for flow cytometry analysis and blood gas analyzer (BGA) analysis.

### Culturing NK Cells (Day 6 to Harvest)

On day 6, 5 L of RPMI-1640 media supplemented with FBS to 10%, Gibco GlutaMAX™ supplement to 1%, Glucose to 1%, and Gibco KnockOut™ Serum Replacement to 0.8% was equilibrated to 50% O2, and 37° C.; rocking at 8 rpm and a 4° angle, in a 10 to 50 L Flexsafe® RM bioreactor bag on a Sartorius wave bioreactor. This completed RPMI (cRPMI) media was used for fed batch and perfusion operations for the remainder of the NK expansion process.

On day 7, a sample was collected and analyzed via flow cytometry to determine pre-stimulation CD56⁺ and CD3⁺ cell counts. The culture volume was reduced to 40 mL and the NK cells were then stimulated a second time by addition of PM21 particles in a ratio that relies on the number NK cells seeded on day 0. Fresh cSCGM was added to raise the incubation volume to 100 mL, and after 15 minutes had elapsed, the culture volume was raised for subsequent culture. Cells were incubated with PM21 particles for another 6 hours, maintained at 37° C., 5% CO2, with agitation. Following re-stimulation, cells were eluted from the CCU and counted via flow cytometry. The entire elution volume was seeded into the Sartorius wave reactor installed on Day 6. A post-seed sample was taken to determine NK cell count. cRPMI media was adjusted until an NK cell concentration of 4.2×10⁵ NK cells/mL was reached. IL-2 was added to the reactor to a concentration of 100 IU/mL. The bioreactor was controlled by a preprogrammed batch record.

On subsequent days, a sample was collected and analyzed by flow cytometry to track NK cell expansion. When necessary, cRPMI media was added to the reactor to reduce NK cell density to 4.2×10⁵ NK cells/mL. IL-2 was added to 100 IU/mL after completing fed batch steps each day. Wave reactor parameters such as gassing, rock rate, and angle were adjusted according to cell density and culture volume. Once the max working volume was achieved, perfusion was initiated at one vessel volumes per day (VVD). After starting perfusion, IL-2 was supplemented to 100 IU/mL in both the reactor volume and the perfusion media every day. Perfusion was increased stepwise according to the NK cell density until harvest was triggered when population doubling time (PDT) reached 40 hours. The total number of expanded NK cells harvested using this commercially viable approach can reach at least about ten billion cells, more preferably at least about one hundred billion cells, and optimally at least about one trillion cells.

### NK Cell Harvesting, Drug Product Fill & Finish

Once harvest was triggered, cells were washed and concentrated with a LOVO® Cell Processing Disposable Kit and a LOVO® Automated Cell Processing system. During concentration, NK cells were exchanged into Plasma-Lyte™ A Injection supplemented with HSA to 10.5 g/L and chilled to 4° C. The final drug substance was sampled and analyzed by flow cytometry before being further diluted to 100×10⁶ NK cells/mL with Plasma-Lyte/HSA buffer. Drug product was established by then combining 1:1 with CryoStor® 10 cryopreservation medium to achieve a final concentration of 50×10⁶ NK cells/mL. Post-formulation, 23 mL of drug product were filled into CryoStore™ freezing bags and placed into a Planer controlled rate freezer for cryopreservation.

### Example 2: Expansion and Characterization of Natural Killer Cells

### Expansion of NK Cells

A small-scale expansion of NK cells was conducted as follows. CD3 Depleted PBMCs were obtained from the Prodigy Centricult™ Unit or cryopreserved CD3 depleted PBMCs were thawed, counted by flow cytometry, and stimulated with PM21 particles at 0.25 mg/mL, before being diluted to 0.3×10⁶ NK cells/mL in cSCGM supplemented with 100 IU/mL of IL-2. On Day 4 of the process, an 80% media exchange is performed with cSCGM supplemented with 100 IU/mL IL-2.

On process days 5 and 6, cells were counted by flow cytometry and underwent 80% media exchanges. On the morning of day 7, a sample was pulled for counting via flow cytometry and the cells were restimulated with PM21 particles for 6 hours, before being diluted to 0.42×10⁶ NK cells/mL in cRPMI media, as previously described. For days 8-13, cells were counted by flow cytometry and diluted to 0.42×10⁶ until a maximum volume of 50 mL was reached. At maximum volume, 100% media exchanges were performed. On Day 13, cells were counted by flow cytometry, and cytotoxicity, surface marker, and characterization panels were completed. Total cell counts for NK cells expanded from fresh apheresis and frozen apheresis samples are depicted in FIG. 1, FIG. 3, FIG. 4, and FIG. 5.

### In-Process Monitoring of NK Cell Count and Viability

Two 100 μL samples of wild type process samples were obtained and incubated with 5 μL of TruStain FcX™ Receptor Blocking solution for 5 minutes. Next, one set of cells was stained with 120 ng CD56 PE and 75 ng CD3 APC for 8-10 minutes, while the isotype control was treated with 120 ng IgG1, κ PE and 75 ng IgG1, κ APC and incubated for the same duration. After incubation, each sample was treated with 0.3 μM DRAQ7 for 2-3 minutes. Stained samples were transferred to TruCount™ Absolute Counting Tubes. Samples were then acquired on the FACS Lyric™ flow cytometer.

### NK Cell-Based Cytotoxicity Assay

Drug Product (DP) samples were obtained from a liquid nitrogen storage tank, thawed, and rested overnight. K562 leukemia cells were counted via NC-200 and 4×10⁶ K562 cells were pulled from culture, split into two tubes, one containing 1×10⁶ K562 cells and another with the remaining 3×10⁶ K562 cells and then washed with the cRPMI media. The tube with 1×10⁶ K562 was labeled “Isotype Control” (Iso) and the tube with 3×10⁶ K562 was labeled “Sample”. After aspirating supernatant down to approximately 100 uL in both tubes and then adding 200 uL of cRPMI media to the sample tube, the Iso tube was incubated with 5 uL, and the Sample tube with 15 uL of TruStain FcX™ Receptor blocking solution. After the overnight rest, the cells were counted via flow cytometry, and 2.4×10⁶ NK cells were serially diluted in a deep-well 96-well plate, and then co-cultured with K562 cells in an effector to target (E:T) ratio of 2:1, 1:1, and 0.5:1. The cells were incubated 37° C. in a 5% CO₂ incubator for approximately 90 minutes. Post co-culture, 100 μL of Annexin V solution (85 μL FITC-Annexin V is diluted into 1615 μL cRPMI media containing IL-2) and 200 μL of DRAQ7™ cell viability solution (40 μL DRAQ-7 stock solution in 4000 μL cRPMI media containing IL-2) were added to the plate and incubated for 15 minutes. Post-incubation, the plate was acquired on the Attune® NxT Acoustic Focusing Cytometer. The results of cytotoxicity assays for NK cells obtained from fresh and frozen apheresis samples are shown in FIG. 2 and FIG. 5. The cytotoxicity of NK cells expanded from manufacturing processing with and without density gradients separation steps is depicted in FIG. 9.

### Quantifying Intracellular Cytokine Populations

Drug product samples were obtained from a liquid nitrogen storage tank, thawed, and rested overnight. After the overnight rest, the cells were counted via flow cytometry, and 5×10⁵ NK cells were combined with 5×10⁵ K562 immortalized cells, pelleted, and resuspended in cRPMI media supplemented with IL-2 and Brefeldin A (BFA). The cells were then centrifuged at 250×g for 2 minutes, and then incubated at 37° C. in a 5% CO₂ incubator for 3.5-4.5 hours.

Next, cells were centrifuged at 250×g for 2 minutes, washed in dPBS and then incubated with 74.65 nmol of DRAQ7 for 5-10 mins. After viability staining, cells were pelleted and then incubated with 5 μL of FcX receptor blocking solution. After incubation, extracellular staining was performed with 0.4 μg of CD56 PE, incubated for 15-25 minutes, and then pelleted.

Cells were then fixed by resuspension in 100 μL of 1× fixation/permeabilization buffer and subsequent incubation for 25-35 minutes. Cells were then permeabilized through two additions and washes in 2 mL 1× permeabilization buffer. Cells were then incubated with 5 μL of FcX receptor blocking solution for 5-9 minutes, before the addition of 5 μL Alexa Fluor™ 647 anti-human IFNγ antibody and 5 μL of PE/Cy7 anti-human TNF-α antibody, and subsequently incubation for 15-25 minutes. Cells were then washed and resuspended in cell staining buffer before acquisition on the Attune® NXT Acoustic Focusing Cytometer. A comparison of TNF-alpha and IFN-gamma levels detected in NK cells expanded from fresh apheresis samples and frozen apheresis samples is shown in FIG. 2. The cytokines were expressed in equivalent levels in NK cells expanded from manufacturing processing with and without density gradients separation steps as shown in FIG. 9.

### In-Process Monitoring of White Blood Cell Surface Markers

Drug product samples were obtained from the liquid nitrogen storage tank, thawed, and rested overnight. After the overnight rest, the cells were counted via flow cytometry, 2×10⁵ NK cells were obtained from the rested culture, pelleted, and washed with 1 mL Cell Staining buffer. Cells were incubated with 5 μL of TruStain™ FcX Receptor Blocking solution, and then stained with FITC anti-human CD16 antibody, PE anti-human CD56 (NCAM) antibody, PerCP/Cy5.5 anti-human CD19 antibody, PE/Cy7 anti-human CD335 (NKp46) antibody, APC anti-human CD314 (NKG2D) antibody. Cell viability was assessed using DRAQ-7 0.3 μM DRAQ7 to determine the number of dead NK cells in the Drug Product samples. Samples were acquired on the FACS Lyric™ flow cytometer. Characterization assays showed that surface marker expression was essentially the same in NK cells expanded from fresh apheresis samples and frozen apheresis samples, as depicted in FIG. 2. White blood cell surface markers were found to be similar in manufacturing processes that included density cell gradient separation and lacked density gradient cell separation steps, as shown in FIG. 9 and FIG. 10.

### Characterization of NK Cell Surface Markers

Drug product samples were obtained from the liquid nitrogen tank, thawed, and rested overnight. After the overnight rest, the NK cells were counted via flow cytometry. 2×10⁵ NK cells were obtained from the rested culture, pelleted, and washed with 1 mL Cell Staining buffer. Cells were incubated with 5 μL of TruStain FcX Receptor Blocking solution, and then stained with FITC anti-human CD16 antibody, PE anti-human CD56 (NCAM) antibody, PerCP/Cy5.5 anti-human CD19 antibody, PE/Cy7 anti-human CD335 (NKp46) antibody, APC anti-human CD314 (NKG2D) antibody. Cell viability was assessed using DRAQ-7 0.3 μM DRAQ7 to determine the number of dead NK cells in the drug product samples. Samples were acquired on the FACS Lyric™ flow cytometer. The results of cell marker assessments can be seen in FIG. 9.

### Results

A split sample study was performed to determine whether drug product from fresh and cryopreserved apheresis are comparable in terms of providing functional natural Killer cells for pharmaceutical use. One full blood volume collection was performed from a human donor, and the leukopheresis product was split immediately for processing. Half of the leukopak was processed within 24 hours of collection as a “fresh sample,” while the other half of the leukopak was immediately processed for cryopreservation and then frozen. After expansion, the cryopreserved apheresis sample yielded more total NK cell numbers than fresh apheresis from the same donor, as shown in FIG. 1. The fresh and the cryopreserved apheresis drug product lots performed comparably in release assays, as shown in FIG. 5. The data indicate that, despite conventional thinking to the contrary, cryopreserved apheresis is a successful means for providing functional NK cells for cell-based drug products. The streamlined workflow process disclosed herein, in combination with frozen apheresis samples from a Universal Donor program, can effectively reduce the time required to process natural killer cells into a cell-based drug product for treating cancer and infectious diseases, such as viruses.

While the foregoing disclosure has been described in some detail by way of illustration and example for purposes of clarity and understanding, it will be clear to one of ordinary skill in the art from a reading of this disclosure that various changes in form and detail can be made without departing from the true scope of the present disclosure and may be practiced within the scope of the appended claims. For example, all constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. For purposes of simplicity those embodiments have not in every case been specifically set forth in so many words herein. It should also be understood that any embodiment or aspect of the present disclosure can be explicitly excluded from the claims, regardless of whether the specific exclusion is recited in the specification.

Although a number of documents are cited herein, this citation does not constitute an admission that any of these documents forms part of the common general knowledge in the art.

All patents, patent applications, websites, other publications or documents, accession numbers and the like cited herein are incorporated by reference in their entirety for all purposes to the same extent as if each individual item were specifically and individually indicated to be so incorporated by reference.

This application claims the benefit of, and relies on the filing date of, U.S. Provisional Application No. 63/689,439, filed 30 Aug. 2024, the entire disclosure of which is herein incorporated by reference.

1. A method of producing expanded natural killer (NK) cells comprising:
(i) thawing a cryopreserved blood sample obtained from a subject;
(ii) obtaining peripheral blood mononuclear cells (PBMCs) from the thawed blood sample without the use of density gradient separation;
(iii) depleting the PBMCs of CD3⁺ cells;
(iv) culturing the PBMCs in a medium with membrane-bound IL-21 to expand NK cells contained in the PBMCs; and
(v) harvesting the expanded NK cells when population doubling reaches a threshold limit.

2. The method of claim 1, wherein the total number of expanded NK cells harvested is at least about ten billion, at least about one hundred billion, or at least about one trillion cells.

3. The method of claim 2, wherein the total number of expanded NK cells is greater than the total number of expanded control NK cells harvested from an equal starting number of cultured PBMCs obtained from a fresh blood sample obtained from the subject and subjected to a method comprising:
(i) obtaining PBMCs from the fresh blood sample using density gradient separation to remove red blood cells;
(ii) depleting the PBMCs of CD3⁺ cells;
(iii) culturing the PBMCs in a medium with membrane-bound IL-21 to expand NK cells contained in the PBMCs; and
(iv) harvesting the expanded control NK cells when population doubling reaches a threshold limit,
wherein the total number of expanded NK cells is greater than the total number of expanded control NK cells after 10, 11, 12, or 13 days of culturing.

4. (canceled)

5. The method of claim 1, wherein harvesting is performed when a population doubling time reaches at least 40 hours.

6. The method of claim 1, wherein the cryopreserved blood sample is a cryopreserved apheresis blood sample, the membrane bound IL-21 is on a membrane particle, and the membrane particle further comprises 4-1BBL.

7. (canceled)

8. (canceled)

9. The method of claim 1, wherein the population doubling time of the expanded NK cells from Day 0 to Day 10 of culture is the same or less than the population doubling time of expanded control NK cells harvested from an equal starting number of cultured PBMCs obtained from a fresh blood sample obtained from the subject and subjected to a method comprising:
(i) obtaining PBMCs from the fresh blood sample using density gradient separation to remove red blood cells;
(ii) depleting the PBMCs of CD3⁺ cells;
(iii) culturing the PBMCs in a medium with membrane-bound IL-21 to expand NK cells contained in the PBMCs; and
(iv) harvesting the expanded control NK cells when population doubling reaches a threshold limit.

10. The method of claim 1, wherein the culturing step further comprises inclusion of IL-2 in the medium and the IL-2 is added at a concentration of 100 IU/mL, and wherein upon harvesting the expanded NK cells have a viability of at least 70%, at least 90%, or at least 95%.

11. (canceled)

12. (canceled)

13. The method of claim 1, wherein the cytotoxicity of the harvested expanded NK cells is equivalent to the cytotoxicity of expanded control NK cells harvested from an equal starting number of cultured PBMCs obtained from a fresh blood sample obtained from the subject and subjected to a method comprising:
(i) obtaining PBMCs from the fresh blood sample using density gradient separation to remove red blood cells;
(ii) depleting the PBMCs of CD3⁺ cells;
(iii) culturing the PBMCs in a medium with membrane-bound IL-21 to expand NK cells contained in the PBMCs; and
(iv) harvesting the expanded control NK cells when population doubling reaches a threshold limit; and
wherein cytotoxicity is measured as percent killing of target cancer cells exposed to the harvested expanded NK cells.

14. (canceled)

15. The method of claim 1, wherein the NK cells are exposed to PM21 particles multiple times during culturing step (iv).

16. A population of expanded natural killer cells produced by the method of claim 1, wherein the expanded natural killer cells obtained from the cryopreserved blood sample exhibit the same percentage of CD16⁺, CD314⁺, and CD355⁺ cell surface markers as expanded natural killer cells obtained from a fresh blood sample.

17. (canceled)

18. The population of expanded natural killer cells according to claim 16, wherein the population comprises at least about ten billion, at least about one hundred billion, or at least about one trillion expanded NK cells.

19. A pharmaceutical composition comprising an effective amount of the expanded natural killer cells produced by the method of claim 1 and a pharmaceutically acceptable carrier.

20. The pharmaceutical composition of claim 19, wherein composition further comprises an anti-infective agent or an anti-cancer agent.

21. The pharmaceutical composition of claim 20, wherein the anti-cancer agent is selected from an antibody, a chemotherapeutic drug, and an immunotherapeutic agent.

22. The pharmaceutical composition of claim 20, wherein the anti-infective agent is selected from an antiviral agent, an antibacterial agent, and an antifungal agent.

23. A method of treating an infection or cancer in a subject in need thereof comprising administering to the subject an effective amount of the pharmaceutical composition of claim 19.

24. (canceled)

25. (canceled)

26. (canceled)

27. (canceled)

28. (canceled)

29. (canceled)

30. (canceled)

31. (canceled)

32. (canceled)

33. (canceled)

34. The method of claim 1, wherein the membrane-bound IL-21 is provided by plasma membrane-bound IL-21 (PM21) particles, exosomes comprising membrane-bound IL-21 (EX21), or feeder cells comprising membrane-bound IL-21 (FC21).

35. The method of claim 34, wherein the PM21 particles further comprise 41-BBL.

36. The method of claim 1, wherein the cryopreserved blood sample is obtained from a leukopak.

37. A kit comprising (i) a pharmaceutical composition comprising an effective amount of the expanded natural killer cells produced by the method of claim 1 and a pharmaceutically acceptable carrier and (ii) instructions for use.
